(19) United States
(12) Patent Application Publication
(10) Pub. No.: US 2017/0254820 A1
(43) Pub. Date: Sep. 7, 2017

(54) Test for the Determination of a Base Concentration

(71) Applicant: AXAGARIUS GmbH & Co. KG, Duren (DE)

(72) Inventors: Dominik Lange, Koln (DE); Jurgen Hoffmann, Duren (DE)

(73) Assignee: Axagarius GmbH & Co. KG, Duren (DE)

(21) Appl. No.: 15/441,550

(22) Filed: Feb. 24, 2017

(30) Foreign Application Priority Data
Mar. 1, 2016 (DE) 102016203335.2

(51) Int. Cl. G01N 33/84; G01N 21/80

(52) U.S. Cl. CPC G01N 33/84; G01N 21/80

(57) ABSTRACT

The subject innovation relates to a device for determining the concentration of a base and here especially hydrogen carbonate, whereby the device contains at least one pH indicator, at least one solid acid, and a mixture of at least one anionic surfactant and one nonionic surfactant. In addition, the subject innovation relates to a method for measuring the base concentration using the device according the subject innovation and a method for increasing the sensitivity of a testing device.

### CROSS-REFERENCE TO RELATED APPLICATION

This application claims priority to German (DE) Patent Application No. DE 102016203335.2, filed on Mar. 1, 2016, the contents of which are incorporated by reference as if set forth in their entirety herein.

### BACKGROUND

In general, dialysates are prepared from an acid (e.g. citric acid, buffer salts, glucose), and an alkaline (e.g. hydrogen carbonate, acetate, lactate) component, as well as water (osmosis water). In contrast to established stable buffer substances the use of hydrogen carbonate is problematic, since being in solution it is not stable. The high physiological compatibility and relating thereto the low probability of side effects is the biggest advantage of hydrogen carbonate as a buffer substance. Normally, the pH value is determined via a pH test strip in order to control the dialysate preparation.

The additional determination of the hydrogen carbonate concentration further increases the patient's security, since wrong hydrogen carbonate concentrations in the dialysate can lead to pathological changes in the patient's blood buffer system. Thus, a hyperosmotic (too highly concentrated) dialysate can lead to hypernatremia or other electrolytic deficiencies, whereas a hypoosmotic (too highly diluted) dialysate can cause a quick hemolysis. It is therefore of vital importance to correctly determine the hydrogen carbonate concentration of the dialysate.

Devices and testing methods to determine the dialysate composition are known in the state of the art. In general, the concentration of ionic components in the dialysate is hereby indirectly determined by the electric conductivity of the dialysate based on the fact that electrolytes are the main dialysate components. Additionally, the pH value of the dialysate is measured.

Here it is of disadvantage that the used devices like conductivity meters and glass pH electrodes use routine maintenance and calibration to ensure an adequate function.

Remarkably, the conductivity measurement exhibits a severe built-in disadvantage, insofar as only a summary representation of the ionic components within the solution can be made. Despite of a correct conductivity there can be a wrong relation of hydrogen carbonate and acid.

Measuring the pH value as a logarithmic measuring method, in addition, shows a too low sensitivity, insofar as only major changes of the relation of hydrogen carbonate and acid can be detected.

As an alternative method for detecting the hydrogen carbonate concentration the U.S. Pat. No. 6,986,999 B2 (Serim Research Corp.) suggests the use of test strips, in which the carrier matrix contains an organic acid, a pH indicator, and an inert dye and the colour change is checked with a colour standard following immersion in the dialysate. These test strips cover a physiologically irrelevantly large measuring range from 18.5 to 74 mEq/L and only allow a distinction of 18.5 vs. 37 vs. 74 mEq/L. Therefore, they are not suitable for a sensitive and significant determination of dialysates.

### SUMMARY

Improved analytic methods and devices for determination of the hydrogen carbonate concentration may be achieved through the subject innovation.

The subject innovation provides a testing device and a testing method for the determination of the hydrogen carbonate concentration in such a way that they are improving at least one of the above mentioned disadvantages.

The subject innovation relates to a device for the determination of the concentration of a base, and especially hydrogen carbonate, whereby the device contains a pH indicator, a solid acid, and a mixture of an anionic and a nonionic surfactant.

In addition, the subject innovation relates to a method for measuring the base concentration using the inventive device and a method for increasing the sensitivity of a testing device.

This objective is achieved according to the subject innovation in that a testing device for the determination of a base concentration is provided whereby the testing device contains a carrier matrix that comprises the following:
a) at least one acid being solid at room temperature;
b) at least one pH indicator having a transition point between the pH value of the solid acid and the base to be determined; and
c) a mixture of at least one anionic and at least one nonionic surfactant.

The testing device according to the subject innovation combines several decisive advantages in comparison to testing devices known from prior art.

As the inventors found out the presence of a mixture of anionic and non-ionic surfactants leads to a significant increase in the sensitivity of the measurement method. Thus, hydrogen carbonate concentrations that differ in only 5 mEq/L units (for example 25, 30, 35, 40, and 45 mEq/L) can be determined in a simple manner Therefore, clinically relevant changes in the hydrogen carbonate concentration can be clearly detected for the first time.

Moreover, the addition of the surfactant mixture allows a restriction of the measuring range to the physiologically/clinically relevant measuring range from 25 to 45 mEq/L hydrogen carbonate.

The resulting measurement represents a distinctive improvement in dialysate analytics since for the first time and in a simple and direct manner it enables a highly sensitive measurement of the dialysate composition. This is possible by that fact that additional dialysate substances in their usual concentration do not disturb the inventive testing method.

The testing device represents a direct and very quick detection method since it is based on a rapid acid-base reaction with direct colour detection. Additionally, the testing device according to the subject innovation is easy to use and to interpret and does not need any additional instruments. Especially in the everyday clinical practice this enhances the compliance since also a user without technical or medical training is able to conduct quick and reliable detection by sight.

The testing device permits a selective determination meaning that it is aimed at alkaline substances only.

The testing device exhibits stability and does not need cooling when used with the usual reagents.

The selection of suitable compounds allows due to the robustness of the testing device a certain variability in the sample to be analysed (for example sample pretreatment is not required) and/or other defined physical parameters during the measurement and still provides reproducible and standardized results.

Moreover, it meets all guidance that a clinically used testing device or method is to fulfill and can therefore also be validated.

The testing device can be produced in a simple and cost-effective way from customary substances.

The testing device can be incorporated into established testing systems without any additional expense.

The person skilled in the art is able to refer to a number of chemical substances with regard to the single components and can therefore specifically adapt the device according to the base to be determined. Especially the selection of the solid acid and the pH indicator permits an application to different bases.

Furthermore, he is able to adjust the measuring range of the concentration by systematically altering the amount of the detection reagents.

### BRIEF DESCRIPTION OF THE DRAWINGS

Moreover, the subject innovation is depicted in the following figure and described below.

FIG. 1 shows a testing device embodied as a test strip with a plastic strip 2 as carrier with a carrier matrix 3 attached to. The carrier matrix contains the detection reagents, such as an organic acid, a pH indicator, and a mixture of an anionic and a nonionic surfactant that are necessary for the determination.

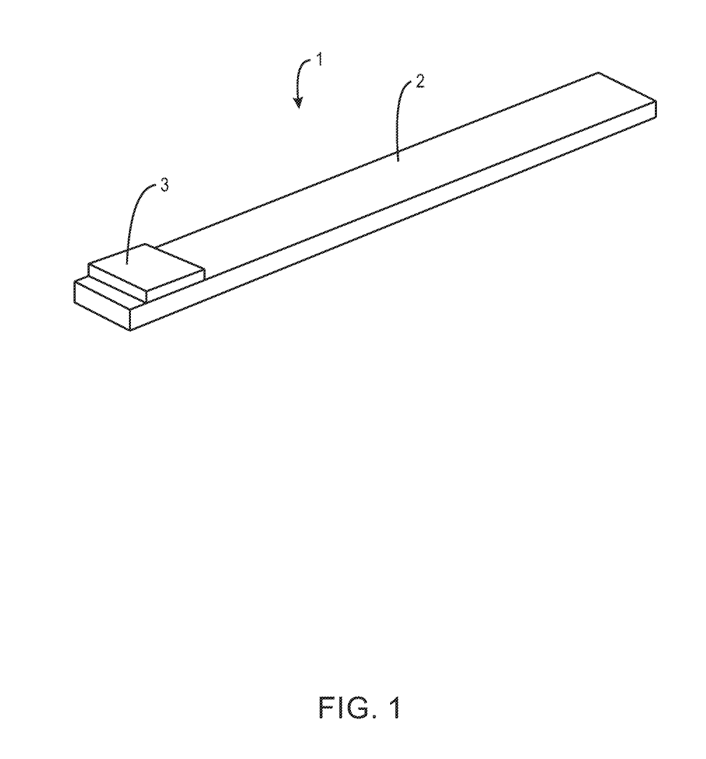

### DETAILED DESCRIPTION OF SPECIFIC EMBODIMENTS

The testing device is based on a titration of the present acid with the base to be determined. The pH indicator having a transition point above the pH value of the acid but below the pH value of the base to be determined is able to display the increasing pH value resulting from the acid-base reaction by an appropriate colour change. The end colour of the pH indicator can be evaluated by different methods. In the simplest form it is checked against a colour on a standard colour chart that has the single colour values assigned to certain base concentrations or corresponding to certain pH values.

The testing device can be used for the determination of all conceivable bases, thus for all alkaline reacting compounds since it is based on the up-titration of the given solid acid.

Thus, anionic bases like hydrogen carbonate anions, cationic bases like [Al³⁺(OH)⁻H₂O₅], monovalent bases like sodium hydroxide, or potassium hydroxide, bivalent bases like calcium hydroxide, or base formers like calcium oxide, barium oxide, or alkali metals can be determined next to neutral bases like NH₃ or amines.

The testing device is used to determine the concentration of hydrogen carbonate.

Used for determining hydrogen carbonate the testing device permits to measure in a narrow measuring range between 25-45 mEq/L hydrogen carbonate and in addition a fine grading of the colour reaction with scale values differing in just 5 mEq/L and that correspond to 25, 30, 35, 40, and 45 mEq/L hydrogen carbonate.

Remarkably, in daily clinical practice the dialysates are produced by dialysis machines that produce a dialysate having a target concentration of 37±2 nmol/l (mEq/L) hydrogen carbonate by controlled addition of a dialysis concentrate. Therefore, the present measuring method is optimally adapted to these hydrogen carbonate concentrations. Considering the machine parameters one can count on deviating hydrogen carbonate concentrations in the range of about 25 to about 45 mEq/L when using dialysis machines known from the state of the art, thus, in the range covered by the inventive testing device.

In another embodiment the testing device provides the use of at least one inert dye. This inert dye matches with the pH indicator and its colour change and enables a better colour change detection by a consistent background colour. Thus, in case of a blue to green transitioning pH indicator it is advantageous to use a yellow inert dye.

According to the subject innovation the inert dye is provided in the same section as the pH indicator. This can be achieved by a number of ways, like for example:
(a) The inert dye can be applied on the carrier itself or can be provided by a coloured carrier
(b) The inert dye can be inserted in the carrier matrix.
(c) The carrier matrix itself can be coloured.
(d) The testing device can have a separate coloured layer below the carrier matrix.

One inert dye may be used. But two, three, or even more inert dyes can also be used to achieve an optimal colouring.

The carrier matrix may contains one inert dye.

In one embodiment of the subject innovation the at least one inert dye is selected from the group containing tartrazine, neozapon yellow, nitrazine yellow, whereby the inert dye is nitrazine yellow.

The solid acid within the testing device serves the reaction with the base to be determined and permits its quantification by change in the pH value. In case it is present as a solid the acid does not evaporate, but remains solid within the carrier matrix and allows long term-stable formulation.

Advantageously, the acid is highly soluble in the liquid solution to be determined, so that the added testing solution immediately dissolves the solid acid and, thereby, enables an efficient acid-base reaction. Highly water-soluble acids like organic acids may be used since most of the testing solutions are aqueous solutions.

When determining an alkaline ampholyte like for example the hydrogen carbonate anion the acid can show a pKa value of about one unit below the pKa value of the ampholyte.

In an embodiment the acid is a physiologically safe substance.

In one embodiment of the subject innovation the at least one solid acid is selected from the group containing citric acid, succinic acid, tartaric acid, phthalic acid, fumaric acid, gluconic acid, malic acid, glycolic acid, malonic acid, glutaric acid, adipic acid, ascorbic acid, amidosulfuric acid, boric acid, diphosphoric acid, and phosphonic acid.

The acid may be tartaric acid.

For the determination of weak bases showing a PK_{B} value between 7.5 and 9 like for example hydrogen carbonate, the at least one acid has a pKs value between 2.9 and 5.6.

One acid may be used. But two, three, or even more acids can also be used, for example to optimally detect a multivalent base having different pK_{B} values.

According to the subject innovation, the testing device uses at least one pH indicator.

The at least one pH indicator has a transition point between pH=3.8 and pH=7.6 for determining weak bases having a PK_{B} value between 7.5 and 9. The use of bromophenol blue is possible.

For determining an ampholyte like hydrogen carbonate having a pKs value of 10.4 and a pKB value of 7.5, the at least one pH indicator can have a transition point of at least pH=4.6 and can be between 3.0 and 4.6. The use of bromophenol blue is possible.

In another embodiment of the subject innovation the at least one pH indicator is selected from the group containing bromophenol blue, methyl orange, tetrabromophenol blue, Congo red, bromocresol green, mitmus, and phenol red.

Depending on the acid present and base to be determined it is also possible to use completely different pH indicators and the person skilled in the art is able to choose from the broad range of indicators available by the pH, pKs, and pK_{B} values present.

One pH indicator may be used. But it is also possible to incorporate two, three, or even more pH indicators for example to broaden the range of measurement.

The mixture of different surfactant classes according to the subject innovation is crucial for the improved analytic of the present measurement method and is due to the mixture of nonionic and anionic surfactants

In one embodiment the nonionic surfactant is selected from the group containing fatty alcohol ethoxylates (FAEO) like Brij35, fatty alcohol propoxylates (FAPO), alkyl glucosides like Tween20, alkyl polyglycosides (APG), octylphenol ethoxylates, and Nonidet-P40.

In an embodiment the nonionic surfactant is Nonidet-P40.

In one embodiment the at least one anionic surfactant is selected from the group containing sodium dodecylsulfate, ammonium dodecylsulfate, sodium lauryl ether sulphate (SLES), sodium myristyl ether sulfate, sodium dioctylsulphosuccinate, perfluorooctane sulphate (PFOS), Perfluorobutane sulfonate, and linear alkylbenzene sulphonates.

In an embodiment the anionic surfactant is sodium dodecylsulfate.

In another embodiment the molar ratio of the anionic and nonionic surfactant is between 10:1 and 1:1, between 5:1 and 1:1, between 4:1 or 3:1 and 1:1, and between 2:1 and 1:1.

Here, in a special way the molar ratio is between 2:1 and 1.5:1 and very especially at 1.6:1.

In one embodiment the detection reagent or several detection reagents or even all detection reagents are immobilized within the carrier matrix.

In one embodiment of the subject innovation the testing device is constructed as test strip or test strap or it is equipped in a way that it is mountable on an integrated testing system.

The test strip can be made of a variety of materials. It is here made of waterproof materials like plastics. Here, the test strip may consist of polyvinyl chloride or polyethylene.

In another embodiment the testing device can have further carrier matrixes.

Different measuring ranges can be covered by one or more carrier matrixes that also aim at the determination of a base concentration.

Advantageously, additional carrier matrixes can also be equipped to determine other target analytes.

Thus, the testing device used for analysis of dialysate can contain a carrier matrix for the determination of the glucose content and/or of the pH value next to the carrier matrix for determining hydrogen carbonate. Appropriate carrier matrixes and detection reagents are known to the person skilled in the art.

Thus, the pH value determination can be made by a pH indicator and the determination of glucose can be made by oxidation by using the enzyme glucose oxidase (GOD; EC 1.1.3.4) releasing hydrogen peroxide. Hydrogen peroxide can then be reduced to water in a downstream colour reaction. This colour reaction is catalysed by a peroxidase (POD)—mostly horseradish peroxidase (EC 1.11.1.7). Accordingly the testing system is called the “GOD/POD test”.

Advantageously, the carrier matrix according to the subject innovation is made of material that permits liquids to pass through. According to the subject innovation this involves especially porous materials that may absorb the liquid und, thus, provide a defined amount of liquid for the reaction with the detection reagents.

The person skilled in the art knows various materials and structures being appropriate as carrier matrixes and he can choose them in a targeted manner depending on the test.

In one embodiment of the subject innovation the carrier matrix is selected from the group containing filter paper, nonwovens, glass fibre, porous polymeric material made of polysulfone, polyester, nylon, nitrocellulose, PVDF, and polycarbonate.

In an embodiment the carrier matrix is filter paper. Filter papers are cost-effective and highly absorbent and can easily be equipped with testing reagents (by soaking and subsequent drying).

In one embodiment the carrier matrix is constructed as a monolayer, in a way that all detecting reagents are contained within this one layer. In an alternative embodiment the carrier matrix can be constructed of two or more layers. The single layers can for example exhibit different absorbencies or absorbing capacities for liquids, thus, the liquid sample can be more specifically absorbed and, in addition, a leakage of the carrier matrix can be prevented. Moreover, this permits a spatial distance between the different detecting reagents, thus, chemically/physically non-compatible detection reagents can be used or the liquid sample entering from the outside sequentially reacts with the detecting reagents during penetrating the single layers.

In addition, the carrier matrix can have an area referred to as “waste pad” that absorbs liquid passing through the carrier matrix. In this area an absorbent pad or a nonwoven, a blotting paper, or a filter paper can be included.

The carrier matrix can be arranged in a shape and depth that it forms a small chromatography column being able to separate possibly disturbing sample components.

In a special embodiment the subject innovation provides a testing device for determining the hydrogen carbonate concentration, whereby the carrier matrix comprises:
a) tartaric acid
b) nitrazine yellow
c) bromophenol blue
d) sodium dodecylsulfate
e) Nonidet-P40

In an embodiment the carrier matrix is applied onto a test strip.

In a second aspect the subject innovation provides a testing method for determining the hydrogen carbonate concentration in a liquid sample by using the inventive testing device that comprises the following steps:
a) soaking the carrier matrix with the liquid sample, and possibly by immersion in the sample;
b) removal of excessive sample material from the carrier matrix, and possibly by extracting the test strip from the sample;
c) an optional incubation of the test strip for at least 5 seconds, and possibly at room temperature;
d) comparing the colour values of the carrier matrix to a colour standard.

In an embodiment the liquid sample is a dialysate.

In a third aspect the subject innovation relates to the usage of a mixture of at least one anionic surfactant and at least one nonionic surfactant, thereby increasing the sensitivity of a carrier matrix-mediated analytical method.

Here it is possible that the surfactants previously disclosed are present in an appropriate molar ratio.

In an embodiment the carrier matrix-mediated analytical method is a method based on an acid base-reaction and may include a carrier matrix that comprises at least one acid, at least one pH indicator, and an inert dye if applicable.

Within the subject innovation, the “testing device” means all carrier-bound tests for medical and non-medical purposes. The carrier-bound tests are detecting reagents embedded in a carrier matrix of carrier that are contacted with the liquid sample. In the presence of target analytes, here the base, the reaction of a liquid sample and of the reagents leads to a detectable signal for example a measurable electric signal or a colour change that can be evaluated visually or by a device for example by transmission photometry, reflexion photometry, or by fluorescence photometry.

According to the subject innovation “bases” refers to all chemical substances that at 25° C. when solved in water reveal a pH value of >7.0. Next to neutral bases like NH₃ these can be anionic bases like hydrogen carbonate anion, cationic bases like [Al³⁺(OH)⁻(H₂O₅)], monovalent bases like sodium hydroxide or potassium hydroxide, bivalent bases like calcium hydroxide, or base formers like calcium oxide, barium oxide, or alkali metals.

According to the subject innovation “solid acid” means an acid exhibiting a solid state of aggregation at room temperature. This can be an organic acid as well as an inorganic acid.

According to the subject innovation the “pH indicator” is comprehended as a substance that changes its colour depending on the pH value.

According to the subject innovation an “inert dye” refers to a dye that at least in the range of the pH according to the test (hence, between the pH value of the present acid and the pH value of the base to be determined) does not or only insignificantly changes its colour, thus, enabling a consistently colour contrasting background.

“Sensitivity” means the magnitude of change in response to a measurement signal divided by the change of the triggering quantity (for example the concentration of the target analyte). The sensitivity of an analytic method corresponds to the slope of the calibration curve.

According to the subject innovation the terms to be distinguished “system precision” (measurement precision) and “methods precision” are defined as follows: The measurement precision is a measure of the variation being caused by the testing device itself or the operating analytical devices. It is determined by multiple analyses of a standard (for example six-fold). The demand on measurement precision depends on the analytical device. In contrast, the methods precision describes the random variation of the analytical results. It is determined by multiply (mostly six-fold) performing the complete analysis, which means starting with weighing, towards sample preparation, up to measuring and evaluating the result (six times weighing of real samples).

The “stability” of the testing device includes storage stability, stability under physical influences like for example warmth, light, or mechanical stress.

The “correctness” is a measure for the deviation of a measured value from the correct value (sometimes referred to as the “true” value) caused by a systematic error. The correctness is determined generally determined by comparison to a working reference (target/actual-comparison), of comparison to an independent, ideally validated method or by the so-called “spiking” of a sample. In case none of the three methods is applicable for certain samples, the following can be valid as a criterion for correctness: Selectivity is proven, linearity is present, and the calibrating curve meets the zero point.

The “detection limit” indicates the smallest concentration (amount) of the analyte in the sample that qualitatively can still be detected (yes/no decision). The “determination limit” is the smallest concentration (amount) of the analyte in the sample that under a given precision and correctness quantitatively can be determined. The underlying mathematical model and the determination methods are described in the DIN 32645.

The “limit of decision” specifies the concentration (amount) that can be detected with probability of 50%. Thus, in a simplified manner the registration limit can be considered as the doubled detection limit.

### EXAMPLE 1

### Generation of a Hydrogen Carbonate Testing Device

A testing solution is produced according to the following recipe:

TABLE 1

| INGREDIENTS | AMOUNT | |
|---|---|---|
| Tartaric acid | 0.50 | g |
| Nitrazine yellow | 0.10 | g |
| Bromophenol blue | 0.03 | g |
| Sodium dodecylsulfate | 0.15 | g |
| Nonidet-P40 | 0.20 | g |
| Ethanol | 15 | mL |
| Water | 85 | mL |

A piece of filter paper is soaked with this solution and dried for 45 seconds at 320° C. By these dried test pads, test strips are produced. The test strip are made of a PVC strip sized 5.5×95 mm as carrier onto which by using hot melt adhesives a test pad of the size 5.5×5 mm is attached to.

### EXAMPLE 2

### Test of a Hydrogen Carbonate Containing Testing Solution

In order to determine the sensitivity of the testing device the test strips were immersed in a dialysate solution (citrate/bicarbonate) containing 25, 30, 35, 40, or 45 mEq/L of hydrogen carbonate. After 5 seconds the test strips were removed from the testing solution and it was waited for another 10-15 seconds until the colour development was complete. The results depicted in the following table show that with increasing hydrogen carbonate concentration the test strips show a colour transition from green to blue and here permit a clear differentiation between the single hydrogen carbonate concentrations of 25, 30, 35, 40, and 45 mEq/L due to the colour.

TABLE 2

| mEq/L bicarbonate | reaction colour |
|---|---|
| 25 | green |
| 30 | light turquoise |
| 35 | turquoise |
| 40 | turquoise blue |
| 45 | blue |

### EXAMPLE 3

### Comparative Study Towards the Influence of Surfactants on the Sensitivity of Testing Paper

The following test papers were produced based on the basic formula depicted in example 1 comprising of pH indicator, inert dye, and acid:

TABLE 3
Without Surfactant

| Ingredients | Amount | |
|---|---|---|
| Tartaric acid | 0.50 | g |
| Nitrazine yellow | 0.10 | g |
| Bromophenol blue | 0.03 | g |
| Ethanol | 15 | mL |
| Water | 85 | mL |

TABLE 4
Nonionic Surfactant

| Ingredients | Amount | |
|---|---|---|
| Tartaric acid | 0.50 | g |
| Nitrazine yellow | 0.10 | g |
| Bromophenol blue | 0.03 | g |
| Nonide-P40 | 0.03 | g |
| Ethanol | 15 | mL |
| Water | 85 | mL |

TABLE 5
Anionic Surfactant

| Ingredients | Amount | |
|---|---|---|
| Tartaric acid | 0.50 | g |
| Nitrazine yellow | 0.10 | g |
| Bromophenol blue | 0.03 | g |
| Sodium dodecylsulfate | 0.15 | g |
| Ethanol | 15 | mL |
| Water | 85 | mL |

TABLE 6
Combination of a Nonioinic and an Anionic
Surfactant

| Ingredients | Amount | |
|---|---|---|
| Tartaric acid | 0.50 | g |
| Nitrazine yellow | 0.10 | g |
| Bromophenol blue | 0.03 | g |
| Sodium dodecylsulfate | 0.15 | g |
| Nonidet-P40 | 0.20 | g |
| Ethanol | 15 | mL |
| Water | 85 | mL |

In order to test the sensitivity of the test papers the test strips were immersed in a dialysate solution (citrate/bicarbonate) containing 25, 30, 35, 40, or 45 mEq/L of hydrogen carbonate. After 5 seconds the test strips were removed from the testing solution and it was waited for another 10-15 seconds until the colour development was complete. The particular colour reactions were visually evaluated and the colour designation for the corresponding concentrations was specified in order to evaluate the efficiency of the single test papers.

### EXAMPLE 4

The following table depicts the reaction colours depending on the hydrogen carbonate concentrations and the surfactant additions:

TABLE 7
Results of the comparative measurement

| | Hydrogen carbonate [mEq/L] | | | | |
|---|---|---|---|---|---|
| Formula | 25 | 30 | 35 | 40 | 45 |
| Without | green | green- | turquoise | turquoise- | blue |
| surfactant | | turquoise | | blue | |
| Nonionic | green | green- | turquoise | turquoise- | blue |
| surfactant | | turquoise | | blue | |
| Anionic | green | green- | turquoise | turquoise- | blue |
| surfactant | | turquoise | | blue | |
| Nonionic | light | light | turquoise | turquoise- | blue |
| surfactant and | yellow- | yellow- | | blue | |
| anionic | green | turquoise | | | |
| surfactant | | | | | |

The special combination of a nonionic and an anionic surfactant shifts the colour reaction in the range of concentration from 25 to 30 mEq/L hydrogen carbonate towards shades more yellow. Thus, the sensitivity of the test paper is increased since a more distinctive differentiation to higher concentrations (from 35 mEq/L) is enabled.

With regard to the complete colour row from 25 to 45 mEq/L the lighter and more yellow shades in the range of 25 to 30 mEq/L hydrogen carbonate generate an overall a more differentiated colour row.

### REFERENCE SIGNS

(1) testing device
(2) plastic strip
(3) carrier matrix

For the person skilled in the art other variants of the subject innovation and their embodiments arise from the preceding disclosure, the figures, and claims.

Terms used in the claims like “comprise”, “have”, “contain”, or the like do not exclude further elements and steps. The use of the indefinite articles does not exclude the plural. A single device can execute the function of several units or devices mentioned. Reference signs specified in the claims are not to be understood as restrictions of the means and steps used.

1. A testing device for the determination of the concentration of a base containing a carrier matrix comprising:
an acid that is solid at room temperature;
a pH indicator with a transition point between a first pH value of the acid at room temperature and a second pH value of a target base being measured; and
a mixture of one or more anionic surfactant and one or more nonionic surfactant.

2. The testing device according to claim 1, wherein the base being measured is selected from the group consisting of hydrogen carbonate, sodium hydroxide, ammonia, and amines.

3. The testing device according to claim 1, wherein the carrier matrix comprises one or more inert dye.

4. The testing device according to claim 3, wherein the one or more inert dye is selected from the group consisting of tartrazine, neozapon yellow, and nitrazine yellow.

5. The testing device according to claim 1, wherein the acid at room temperature is selected from the group consisting of citric acid, succinic acid, tartaric acid, phthalic acid, fumaric acid, gluconic acid, malic acid, glycolic acid, malonic acid, glutaric acid, adipic acid, ascorbic acid, amidosulfuric acid, boric acid, diphosphoric acid, and phosphonic acid.

6. The testing device according to claim 1, wherein the pH indicator has a pKs value between 3.8 and 7.6 and is selected from the group consisting of bromophenol blue, methyl orange, tetrabromophenol blue, Congo red, bromocresol green, mitmus, and phenol red.

7. The testing device according to claim 1, wherein the one or more nonionic surfactant is selected from the group consisting of fatty alcohol ethoxylates (FAEO) like Brij35, fatty alcohol propoxylates (FAPO), alkyl glucosides like Tween20, alkyl polyglycosides (APG), octylphenol ethoxylates, and Nonidet-P40.

8. The testing device according to claim 1, wherein the one or more anionic surfactant is selected from the group consisting of sodium dodecylsulfate, ammonium dodecylsulfate, sodium lauryl ether sulphate (SLES), sodium myristyl ether sulfate, sodium dioctylsulphosuccinate, perfluorooctane sulphate (PFOS), Perfluorobutane sulfonate, and linear alkylbenzene sulphonates.

9. The testing device according to claim 1, wherein the molar ratio of the one or more anionic surfactant and the one or more nonionic surfactant is between 10:1 and 1:1.

10. The testing device according to claim 1, wherein the device is in the form of a test strip, a test strap, or is mountable on an integrated testing system.

11. The testing device according to claim 1, wherein the carrier matrix is a porous material that permits liquids to pass through and is selected from the group comprising filter paper, nonwovens, glass fibre, porous polymeric material made of polysulfone, polyester, nylon, nitrocellulose, PVDF, and polycarbonate.

12. The testing device for the determination of a hydrogen carbonate concentration according to claim 1, wherein the device is a test strip having a carrier matrix that comprises tartaric acid, nitrazine yellow, bromophenol blue, sodium dodecylsulfate, and Nonidet-P40.

13. A testing method for determining the hydrogen carbonate concentration in a liquid sample using a testing device, the method comprising:
soaking the carrier matrix with the liquid sample by immersion in the sample;
removing excessive sample material from the carrier matrix by extracting the test strip from the sample;
comparing the colour values of the carrier matrix to a colour standard, whereby the

14. The method of claim 13, wherein the liquid sample is a dialysate.

15. The method of claim 13, comprising incubating the test strip for at least 5 seconds at room temperature.

16. The method of claim 13, comprising using a mixture of one or more anionic surfactant and one or more nonionic surfactant.

17. The method of claim 13, wherein the base being measured is selected from the group consisting of hydrogen carbonate, sodium hydroxide, ammonia, and amines.

18. The method of claim 13, wherein the carrier matrix comprises one or more inert dye.

19. The method of claim 18, wherein the one or more inert dye is selected from the group consisting of tartrazine, neozapon yellow, and nitrazine yellow.

20. The method of claim 13, wherein the acid at room temperature is selected from the group consisting of citric acid, succinic acid, tartaric acid, phthalic acid, fumaric acid, gluconic acid, malic acid, glycolic acid, malonic acid, glutaric acid, adipic acid, ascorbic acid, amidosulfuric acid, boric acid, diphosphoric acid, and phosphonic acid.
